(19) United States
(12) Patent Application Publication
(10) Pub. No.: US 2025/0281906 A1
(43) Pub. Date: Sep. 11, 2025

(54) METHOD FOR PRODUCING TITANIUM-CONTAINING SILICON OXIDE, METHOD FOR PRODUCING EPOXIDE, AND TITANIUM-CONTAINING SILICON OXIDE

(71) Applicant: SUMITOMO CHEMICAL COMPANY, LIMITED, Chuo-ku, Tokyo (JP)

(72) Inventor: Motoshi MATOBA, Tokyo (JP)

(21) Appl. No.: 18/859,285

(22) PCT Filed: Mar. 15, 2023
(86) PCT No.: PCT/JP2023/010034

§ 371 Date: Oct. 23, 2024

(30) Foreign Application Priority Data
Apr. 25, 2022 (JP) 2022-071921

(51) Int. Cl. B01J 21/06; B01J 35/63; B01J 35/64; B01J 37/00; B01J 37/04; C07D 301/19

(52) U.S. Cl. CPC B01J 21/063; B01J 35/638; B01J 35/647; B01J 37/0018; B01J 37/04; C07D 301/19

(57) ABSTRACT

A method for producing an epoxide using a titanium-containing silicon oxide catalyst that includes a salt at a predetermined concentration.

### TECHNICAL FIELD

The present invention relates to a method for producing a titanium-containing silicon oxide, a method for producing an epoxide from an olefin in the presence of the titanium-containing silicon oxide, and the titanium-containing silicon oxide.

### BACKGROUND ART

A method for producing an epoxide from a hydroperoxide and an olefin in the presence of a catalyst is known. As a catalyst used in this method, a titanium-containing silicon oxide is described in, for example, Patent Document 1.

### PRIOR ART DOCUMENT

### Patent Document

Patent Document 1: CN 102807537 B

### SUMMARY OF INVENTION

### Technical Problem

In a reaction of generating an epoxide from an olefin and a hydroperoxide, a method capable of producing an epoxide in a high yield has been desired.

### Solution to Problem

The present invention relates to the following, but is not limited thereto.

<1>

A titanium-containing silicon oxide satisfying all conditions 1 to 5:
condition 1: an average pore size is 10 Å or more;
condition 2: pores of 80% or more of a total pore volume each have a pore size of 5 to 200 Å;
condition 3: the total pore volume is 0.2 cm³/g or more;
condition 4: the titanium-containing silicon oxide is obtained by using a quaternary ammonium ion represented by formula I as a template agent and thereafter removing the template agent by a solvent extraction operation:

[NR¹R²R³R⁴]≥ I
wherein R¹ represents a C₂₋₃₆ hydrocarbon group, and R² to R⁴ each independently represent a C₁₋₆ hydrocarbon group;
condition 5: a ratio of an amount of substance of a salt S to an amount of substance of titanium atoms in the titanium-containing silicon oxide is 0.004 to 10, and the salt S is at least one selected from the group consisting of ammonium salts, alkali metal salts, and alkaline earth metal salts.
   <2>

The titanium-containing silicon oxide according to <1>, wherein the ammonium salt is ammonium chloride.

<3>

Use of the titanium-containing silicon oxide according to <1> or <2> for producing an epoxide from an olefin.

<4>

A method for producing a titanium-containing silicon oxide, comprising the following steps:
a step of mixing a silicon source, a template agent, and a solvent to obtain a solid including a silicon oxide and the template agent (raw material mixing step);
a step of removing the template agent from the solid obtained in the raw material mixing step to obtain a solid including the silicon oxide (template agent removing step);
a step of contacting the solid obtained in the template agent removing step with a silylating agent to obtain a solid including a silylated silicon oxide (silylation step);
a step of introducing titanium into the system (titanium introducing step); and
a step of introducing or removing a salt S or a precursor thereof into the system to adjust a molar concentration of the salt S or a precursor thereof relative to an amount of substance of titanium atoms into the system, wherein the salt S is at least one selected from the group consisting of ammonium salts, alkali metal salts, and alkaline earth metal salts (salt concentration adjusting step).
   <5>

A method for producing an epoxide, comprising a step of reacting an olefin with a hydroperoxide in the presence of the titanium-containing silicon oxide according to <1> or <2>.

<6>

The method according to <5>, wherein the olefin is propylene.

<7>

The method according to <5> or <6>, wherein the hydroperoxide is cumene hydroperoxide.

### Advantageous Effect of Invention

According to one aspect of the present invention, in a reaction of generating an epoxide from an olefin and a hydroperoxide, a method for producing an epoxide in a high yield, and the like are provided.

### DESCRIPTION OF EMBODIMENTS

### Definitions

Herein, a term “solution” includes not only a homogeneous liquid but also a colloidal or suspension-like mixture, and further includes a gas-liquid mixture.

Herein, a term “α-olefin” means a hydrocarbon having a carbon-carbon unsaturated double bond at an α position.

Herein, a term “C_{X-Y} hydrocarbon group” means a hydrocarbon group having X to Y carbon atoms.

Herein, all the numbers disclosed are approximate values whether a word “about” or “roughly” is used or not in connection therewith. They may vary by 1%, 2%, 5%, or sometimes to 20%. Whenever a numerical range accompanied by a lower limit R^{L} and an upper limit RU is disclosed, an optional number included in the range is particularly disclosed. Especially, the following number within the range is disclosed. R=R^{L}+k*(RU−RL) (wherein k is a variable in the range of 1% to 100%, which increases by 1%, that is, k is 1%, 2%, 3%, 4%, 5%, . . . , 50%, 51%, 52%, . . . , 95%, 96%, 97%, 98%, 99%, or 100%). Furthermore, an optional numerical range defined by two numbers of R described above is also particularly disclosed.

The description “lower limit to upper limit” representing a numerical range represents “lower limit or more and upper limit or less”, and the description “upper limit to lower limit” represents “upper limit or less and lower limit or more”. In other words, these descriptions each represent a numerical range including a lower limit and an upper limit, but in one aspect, one or both of the upper limit and the lower limit may be excluded, that is, “lower limit to upper limit” may represent “more than lower limit and upper limit or less”, “lower limit or more and less than upper limit”, or “more than lower limit and less than upper limit”. Likewise, “xx or more” may represent “more than xx”, and “xx or less” may represent “less than xx”.

### Titanium-Containing Silicon Oxide

Herein, a titanium-containing silicon oxide refers to a compound in which a part of Si in a porous silicate (SiO₂) is substituted by Ti. The compound has a bond represented by —Si—O—Ti.

The titanium-containing silicon oxide of the present invention satisfies all conditions 1 to 5.

Condition 1 is that an average pore size is 10 Å or more.

Condition 2 is that pores of 80% or more of a total pore volume each have a pore size of 5 to 200 Å.

Condition 3 is that the total pore volume is 0.2 cm³/g or more. In this regard, the total pore volume means a pore volume per 1 g of the titanium-containing silicon oxide.

The measurement regarding conditions 1 to 3 can be carried out by a usual method using a physical adsorption method for a gas such as nitrogen or argon. For example, the measurement is carried out in accordance with the method described in the Examples.

The average pore size is preferably 20 Å or more from the viewpoint of diffusibility. From the viewpoint of effective area, pores of 90% or more of the total pore volume each preferably have a pore size of 5 to 200 Å. The total pore volume is preferably 0.5 cm³/g or more.

Condition 4 is that the titanium-containing silicon oxide is obtained by using a quaternary ammonium ion represented by formula I as a template agent and thereafter removing the template agent by a solvent extraction operation:

[NR¹R²R³R⁴]⁺ I
wherein R¹ represents a C₂₋₃₆ hydrocarbon group, and R² to R⁴ each independently represent a C₁₋₆ hydrocarbon group.

Condition 4 will be described in detail together with the description of a method for producing the titanium-containing silicon oxide (particularly, raw material mixing step, template agent removing step), and the like.

Condition 5 is that a ratio of an amount of substance of a salt S to an amount of substance of titanium atoms in the titanium-containing silicon oxide is 0.004 to 10, and the salt S is at least one selected from the group consisting of ammonium salts, alkali metal salts, and alkaline earth metal salts. In this regard, in the ammonium salts, not only a narrow-sense salt of an ammonium ion (NH₄⁺) and an anion but also a salt of a substituted ammonium ion ([NR¹R²R³R⁴]⁺) and an anion is included. The salt S is preferably a substituted or unsubstituted ammonium salt, more preferably substituted or unsubstituted ammonium chloride, and still more preferably unsubstituted ammonium chloride. In the above-mentioned ratio, the lower limit is preferably 0.01 or more, and more preferably 0.1 or more. In the above-mentioned ratio, the upper limit is 4 or less, and more preferably 1 or less.

Condition 5 will be described in detail together with the description of a method for producing the titanium-containing silicon oxide (particularly, salt concentration adjusting step), and the like.

### Production Method

The method for producing a titanium-containing silicon oxide according to one aspect of the present invention includes a raw material mixing step, a template agent removing step, a silylation step, a titanium introducing step, and a salt concentration adjusting step.

### Raw Material Mixing Step

The raw material mixing step is a step of mixing a silicon source, a template agent, and a solvent to obtain a solid including a silicon oxide and a template agent and also sometimes referred to as Step A.

The “silicon source” means a silicon oxide and a silicon oxide precursor. The silicon oxide precursor means a compound in which a part or the whole of the silicon oxide precursor becomes a silicon oxide by reacting the silicon oxide precursor with water.

Examples of the silicon oxide include amorphous silica. Examples of the silicon oxide precursor include an alkoxysilane, an alkyltrialkoxysilane, a dialkyldialkoxysilane and a 1,2-bis(trialkoxysilyl)alkane. Examples of the alkoxysilane include tetramethyl orthosilicate, tetraethyl orthosilicate and tetrapropyl orthosilicate. Examples of alkyltrialkoxysilane include trimethoxy(methyl)silane. Examples of the dialkyldialkoxysilane include dimethoxydimethylsilane. As the silicon source, a single one may be used, or several kinds thereof may be used in combination.

When the silicon oxide precursor is used as the silicon source, water is preferably used as a part or the whole of the solvent in Step A. When the silicon oxide precursor is mixed with water, a part or the whole of the silicon oxide precursor is changed to the silicon oxide.

The template agent means a substance capable of forming a pore structure in the titanium-containing silicon oxide. As the template agent, a quaternary ammonium compound having a quaternary ammonium ion represented by formula I can be preferably used.

[NR¹R²R³R⁴]⁺ I
wherein R¹ represents a C₂₋₃₆ hydrocarbon group, and R² to R⁴ each independently represent a C₁₋₆ hydrocarbon group.

In formula I, R¹ is a C₂₋₃₆ hydrocarbon group, may be linear or branched, and may be aliphatic or aromatic. Preferably, it is a C₁₀₋₂₂ hydrocarbon group. R² to R⁴ are each independently a C₁₋₆ hydrocarbon group, preferably aliphatic, and may be linear or branched. R² to R⁴ are more preferably all methyl groups.

Examples of the quaternary ammonium ions represented by formula I include cations, such as tetraethylammonium, tetrapropylammonium, tetrabutylammonium, decyltrimethylammonium, dodecyltrimethylammonium, hexadecyltrimethylammonium, octadecyltrimethylammonium, eicosyltrimethylammonium, behenyltrimethylammonium, and benzyltrimethylammonium.

Examples of the compounds containing a quaternary ammonium ion represented by formula I include tetraethylammonium hydroxide, tetrapropylammonium hydroxide, tetrabutylammonium hydroxide, decyltrimethylammonium hydroxide, decyltrimethylammonium chloride, decyltrimethylammonium bromide, dodecyltrimethylammonium hydroxide, dodecyltrimethylammonium chloride, dodecyltrimethylammonium bromide, hexadecyltrimethylammonium hydroxide, hexadecyltrimethylammonium chloride, hexadecyltrimethylammonium bromide, octadecyltrimethylammonium hydroxide, octadecyltrimethylammonium chloride, octadecyltrimethylammonium bromide, eicosyltrimethylammonium hydroxide, eicosyltrimethylammonium chloride, eicosyltrimethylammonium bromide, behenyltrimethylammonium hydroxide, behenyltrimethylammonium chloride, and behenyltrimethylammonium bromide.

The mixing of the silicon source and the template agent is carried out in the presence of the solvent. Examples of the solvent include water and an alcohol. Examples of the alcohol include methanol, ethanol, 1-propanol and 2-propanol. Two or more kinds of solvents may be mixed to be used.

The solid including the silicon oxide and the template agent can be obtained through Step A. Step A usually includes a solvent removing step. The obtained solid including the silicon oxide and the template agent can be taken out by filtering, decantation, drying, centrifugal separation, a combination thereof, and the like. The mixing in Step A is preferably carried out from 0 to 300° C. over 30 minutes to 1000 hours. In one aspect, the mixing may be carried out from 20 to 100° C., the mixing may be carried out at the boiling point of the solvent, the mixing may be carried out from 20 to 60° C., and the mixing may be carried out from 20 to 40° C. In one aspect, the mixing may be carried out over 30 minutes to 24 hours and the mixing may be carried out over 2 to 24 hours. In addition, the stirring can also be carried out during mixing.

### Template Agent Removing Step

The template agent removing step is a step of removing the template agent from the solid obtained in Step A to obtain the solid including the silicon oxide and also sometimes referred to as Step B. By carrying out Step B, the solid which does not include the template agent or does not substantially include the template agent can be obtained.

The content of the template agent in the solid obtained in Step B is preferably 10% by mass or less and more preferably 1% by mass or less

The removal of the template agent can be achieved by calcining the solid including the template agent from 300 to 800° C. under air or extracting it with a solvent. The template agent is preferably removed by extracting.

Whitehurst et al. have reported a technique of extracting a template agent with a solvent (see U.S. Pat. No. 5,143,879). Any solvent may be used as long as the solvent can dissolve the compound used as the template agent. Generally, compounds which are in a liquid state at normal temperature and have 1 to 12 carbon atoms or a mixture of two or more kinds of these compounds can be used. Examples of the suitable solvent include an alcohol, a ketone, an acyclic ether or ester and a cyclic ether or ester. Examples of the alcohol include methanol, ethanol, ethylene glycol, propylene glycol, 1-propanol, 2-propanol, 1-butanol and octanol. Examples of the ketone include acetone, diethyl ketone, methyl ethyl ketone and methyl isobutyl ketone. Examples of the ether include diisobutyl ether and tetrahydrofuran. Examples of the ester include methyl acetate, ethyl acetate, butyl acetate and butyl propionate.

As examples of alcohols, from the viewpoint of dissolution ability of the template agent, when the template agent is a compound including the quaternary ammonium ion, alcohols are preferable, and among those, methanol is more preferable. The mass ratio of the solvent to the solid including the template agent is usually from 1 to 1000 and preferably from 5 to 300. An acid or a salt thereof may be added to these solvents in order to improve the extraction effect. Examples of the acid used include an inorganic acid such as hydrochloric acid, sulfuric acid, nitric acid and hydrobromic acid, or an organic acid such as formic acid, acetic acid and propionic acid. Examples of the salt thereof include an alkali metal salt, an alkaline earth metal salt and an ammonium salt. The concentration of the acid or the salt thereof added in the solvent is preferably 30% by mass or less and even more preferably 15% by mass or less.

Examples of the template agent removing method include a method in which the solvent is sufficiently mixed with the solid including the template agent and then the liquid phase part is separated through a method such as filtering, decantation, drying, centrifugal separation, and a combination thereof. This operation may be repeated multiple times. It is also possible to extract the template agent through a method in which a container such as a column is filled with the solid including the template agent and an extraction solvent is passed therethrough. The extraction temperature is preferably from 0 to 200° C. and even more preferably from 20 to 100° C. When the boiling point of the extraction solvent is low, the extraction may be carried out under pressure.

Carrying out a treatment such as an ion exchange if necessary, allows the template agent in the solution obtained by the extract treatment to be recovered to be reused as the template agent in Step A. In addition, similarly, the purification by a normal distillation operation and the like also allows the extraction solvent to be reused.

### Silylation Step

The silylation step is a step of contacting the solid obtained in Step B with a silylating agent to obtain a solid including a silylated silicon oxide and also sometimes referred to as Step C. By carrying out Step C, the silicon oxide included in the solid obtained in Step B is silylated.

The silylation may be carried out through a gas phase method in which a gaseous silylating agent is contacted and reacted with the solid obtained in Step B or may be carried out through a liquid phase method in which a silylating agent is contacted and reacted with the solid in a solvent. In one aspect of the present invention, the liquid phase method is preferable. Usually, when the silylation is carried out through the liquid phase method, a hydrocarbon is suitably used in Step C as a solvent. When the silylation is carried out through the liquid phase method, the drying may be carried out thereafter.

The silylating agent is a silicon compound having reactivity with the solid and a hydrolytic group is bonded to silicon. Examples of the hydrolytic group bonded to silicon include hydrogen, halogen, an alkoxy group, an acetoxy group and an amino group. In the silylating agent, the hydrolytic group bonded to silicon is preferably one. In addition, at least one or more groups selected from the group consisting of an alkyl group, an alkenyl group such as a vinyl group, an aryl group such as a phenyl group, a halogenated alkyl group, a siloxy group, and the like are bonded to silicon.

Examples of the silylating agent include an organic silane, an organic silylamine, an organic silylamide and a derivative thereof, and an organic silazane.

Examples of the organic silane include chlorotrimethylsilane, dichlorodimethylsilane, chlorobromodimethylsilane, nitrotrimethylsilane, chlorotriethylsilane, iododimethylbutylsilane, chlorodimethylphenylsilane, chlorodimethylsilane, dimethyl-n-propylchlorosilane, dimethylisopropylchlorosilane, tert-butyldimethylchlorosilane, tripropylchlorosilane, dimethyloctylchlorosilane, tributylchlorosilane, trihexylchlorosilane, dimethylethylchlorosilane, dimethyloctadecylchlorosilane, n-butyldimethylchlorosilane, bromomethyldimethylchlorosilane, chloromethyldimethylchlorosilane, 3-chloropropyldimethylchlorosilane, dimethoxymethylchlorosilane, methylphenylchlorosilane, methylphenylvinylchlorosilane, benzyldimethylchlorosilane, diphenylchlorosilane, diphenylmethylchlorosilane, diphenylvinylchlorosilane, tribenzylchlorosilane, methoxytrimethylsilane, dimethoxydimethylsilane, trimethoxymethylsilane, ethoxytrimethylsilane, diethoxydimethylsilane, triethoxymethylsilane, trimethoxyethylsilane, dimethoxydiethylsilane, methoxytriethylsilane, ethoxytriethylsilane, diethoxydiethylsilane, triethoxyethylsilane, methoxytriphenylsilane, dimethoxydiphenylsilane, trimethoxyphenylsilane, ethoxytriphenylsilane, diethoxydiphenylsilane, triethoxyphenylsilane, dichlorotetramethyldisiloxane, 3-cyanopropyldimethylchlorosilane, 1,3-dichloro-1,1,3,3-tetramethyldisiloxane, and 1,3-dimethoxy-1,1,3,3-tetramethyldisiloxane.

Examples of the organic silylamine include N-(trimethylsilyl)imidazole, N-(tert-butyldimethylsilyl)imidazole, N-(dimethylethylsilyl)imidazole, N-(dimethyl-n-propylsilyl)imidazole, N-(dimethylisopropylsilyl)imidazole, N-(trimethylsilyl)-N,N-dimethylamine, N-(trimethylsilyl)-N,N-diethylamine, N-(trimethylsilyl)pyrrole, N-(trimethylsilyl)pyrrolidine, N-(trimethylsilyl)piperidine, 1-cyanoethyl(diethylamino)dimethylsilane, and pentafluorophenyldimethylsilylamine.

Examples of the organic silylamide and the derivative thereof include N,O-bis(trimethylsilyl)acetamide, N,O-bis(trimethylsilyl)trifluoroacetamide, N-(trimethylsilyl)acetamide, N-methyl-N-(trimethylsilyl)acetamide, N-methyl-N-(trimethylsilyl)trifluoroacetamide, N-methyl-N-(trimethylsilyl)heptafluorobutylamide, N-(tert-butyldimethylsilyl)-N-trifluoroacetamide, and N,O-bis(diethylhydrosilyl)trifluoroacetamide.

Examples of the organic silazane include 1,1,1,3,3,3-hexamethyldisilazane, heptamethyldisilazane, 1,1,3,3-tetramethyldisilazane, 1,3-bis(chloromethyl)-1,1,3,3-tetramethyldisilazane, 1,3-divinyl-1,1,3,3-tetramethyldisilazane, and 1,3-diphenyl-1,1,3,3-tetramethyldisilazane.

Further examples of silylating agent include N-methoxy-N,O-bis(trimethylsilyl)trifluoroacetamide, N-methoxy-N,O-bis(trimethylsilyl)carbamate, N,O-bis(trimethylsilyl)sulfamate, trimethylsilyl trifluoromethanesulfonate, and N,N′-bis(trimethylsilyl)urea.

The silylating agent is preferably an organic silazane and more preferably 1,1,1,3,3,3-hexamethyldisilazane.

The solid which is obtained in Step B and includes the silicon oxide contacts with the silylating agent, thereby silylating the silicon oxide. It is assumed that, in at least a part of the solid including the silicon oxide, a silyl group is introduced into an OH group on its surface to make it hydrophobized, however, the present invention is not limited to this theory.

### Titanium Introducing Step

The titanium introducing step is a step of introducing titanium into the system and also sometimes referred to as Step D. The expression “into the system” means “into the reaction system” in a method for producing a titanium-containing silicon oxide, and it means, for example, “into the reaction” before Step A, in Step A, between Step A and Step B, in Step B, between Step B and Step C, in Step C, and after Step C.

Titanium is introduced into the system, thereby mixing the silicon oxide with the titanium source to introduce a bond represented by —Si—O—Ti into the silicon oxide.

Titanium may be introduced into the silicon oxide by mixing and contacting the silicon oxide with the titanium source in the liquid phase, and titanium may be introduced into the silicon oxide by mixing and contacting the silicon oxide with gas including the titanium source.

When the mixing is carried out in the liquid phase, examples of the solvent include water and an alcohol and, for example, the above-described solvents for Step A can be used. Examples of the mixing temperature include from 0 to 60° C. Examples of the mixing time include from 1 minute to 24 hours.

When the mixing is carried out in the gas phase, the titanium source can be gasified and mixed. Examples of the mixing temperature include from 100 to 500° C. Examples of the mixing time include from 1 minute to 24 hours. The mixing may be carried out at normal pressure and, for example, may be carried out at 10 to 1000 kPa (absolute pressure).

Titanium may be introduced in any timing of before Step A, in Step A, between Step A and Step B, in Step B, between Step B and Step C, in Step C and after Step C. Titanium may be introduced at two or more timings of the timings described above.

Titanium is preferably introduced before starting Step C, titanium is more preferably introduced in at least one or more selected from the group consisting of before Step A, in Step A and between Step B and Step C, and titanium is even more preferably introduced before Step A or in Step A.

When titanium is introduced before Step A, the titanium source is mixed with the silicon source, the template agent or the solvent before mixing in Step A.

When titanium is introduced in Step A, the silicon source, the titanium source and the template agent are mixed in Step A.

Titanium may be introduced by contacting the solid obtained in Step A with the titanium source after finishing Step A and before starting Step B.

Titanium may be introduced by contacting the solid obtained in Step B with the titanium source after finishing Step B and before starting Step C.

Examples of the titanium source include a titanium alkoxide, a chelate type titanium complex, a titanium halide and a sulfate including titanium. Examples of the titanium alkoxide include tetramethyl titanate, tetraethyl titanate, tetrapropyl titanate, tetraisopropyl titanate, tetrabutyl titanate, tetraisobutyl titanate, tetra(2-ethylhexyl)titanate and tetraoctadecyl titanate. Examples of the chelate type titanium complex include titanium(IV)oxyacetylacetonate and titanium(IV)diisopropoxybisacetylacetonate. Examples of the titanium halide include titanium tetrachloride, titanium tetrabromide and titanium tetraiodide. Examples of the sulfate including titanium include titanyl sulfate.

### Salt Concentration Adjusting Step

The salt concentration adjusting step is a step of introducing or removing a salt S or a precursor thereof into the system to adjust a molar concentration of the salt S or a precursor thereof relative to an amount of substance of titanium atoms into the system and also sometimes referred to as step E. The expression “into the system” means “into the reaction system” in the method for producing a titanium-containing silicon oxide, and it means, for example, “into the system” before step A, in Step A, between Step A and Step B, in Step B, between Step B and Step C, in Step C, and after Step C. The salt S is at least one selected from the group consisting of ammonium salts, alkali metal salts, and alkaline earth metal salts. In this regard, in the ammonium salts, not only a narrow-sense salt of an ammonium ion (NH₄⁺) and an anion but also a salt of a substituted ammonium ion ([NR¹R²R³R⁴]⁺) and an anion is included.

The concentration of the salt S or a precursor thereof into the system can be appropriately adjusted according to the composition of the desired object, and preferably, it can be adjusted so that the ratio of the molar concentration of the salt S to the amount of substance of titanium atoms in the titanium-containing silicon oxide may become 0.004 to 10.

Preferred examples of the method for introducing or removing the salt S or a precursor thereof into the system include, but not limited to, the following methods.

Method for Introducing Salt S or Precursor Thereof in Raw Material of Step a, Step B, and/or Step C

The introduction method is, for example, a method in which the salt S is added to the raw material of each step and they are mixed. It is also possible that a plurality of salt S precursors are added to the raw material of each step and they are reacted in situ to introduce a salt produced. The introduction may be carried out over multiple steps.

Method for Removing the Above Salt or Precursor Thereof from Reaction Solution into the System

The removal method is, for example, a method selected from filtration, distillation, fractionation, recrystallization, sublimation method, chromatography, ion exchange, adsorption separation, extraction separation, and optional combinations of these. The removal may be carried out over multiple steps.

Method for Introducing the Salt into Solid Obtained in Step a, Step B, and/or Step C

Examples of the introduction methods include an impregnation method in which a solution containing the salt S or a precursor thereof dissolved in an alcohol solvent, such as methanol or ethanol, and/or water is introduced to the solid by a pore-filling method, an immersion method in which the solid is immersed in the solution to perform introduction, a spraying method in which the solution is sprayed onto the solid to perform introduction, and a method in which vapor obtained by vaporizing the salt S or a precursor thereof or a gas containing the salt S or a precursor thereof is brought into contact with the solid. At this stage, it is also possible that a plurality of salt S precursors are added and reacted in situ to introduce a salt produced. The introduction may be carried out over multiple steps.

Method for Removing Salt S or Precursor Thereof from Solid Obtained in Step a, Step B, and/or Step C

Examples of the removal methods include a removal method using a sublimation method or thermal decomposition in an environment of high temperature or reduced pressure or both of them, sieving utilizing particle size differences, centrifuging, and air flow classification. A method in which a solvent having high dissolving power for the salt S or a precursor thereof is brought into contact with the solid to remove the salt S or a precursor thereof in the solid is also employable. At this stage, pretreatment to convert the salt S or a precursor thereof to a salt that is easy to dissolve in a specific solvent may be carried out as pretreatment. The removal may be carried out over multiple steps.

### Type of Salt

A salt is a compound wherein a negative ion (anion) derived from an acid and a positive ion (cation) derived from a base are ionically bonded. The salt S suitable for the object of the present invention is an optional combination of the following cation and anion: the cation is one or more selected from an ammonium ion ([NR⁵R⁶R⁷R⁸]⁺; R⁵ to R⁸ each independently represent a C₁₋₆ hydrocarbon group or H), an alkali metal ion (particularly, Li⁺, Na⁺, K⁺, Rb⁺, Cs⁺), and an alkaline earth metal ion (particularly, Mg²⁺, Ca²⁺, Sr²⁺, Ba²⁺). The anion is one or more selected from a halide ion (particularly, Cl⁻, Br⁻, I⁻), a nitrate ion (NO₃⁻), a sulfate ion (SO₄²⁻), a phosphate ion (PO₄³⁻), a hydroxide ion (OH⁻), a carbonate ion (CO₃²⁻), a bicarbonate ion (HCO₃⁻), an organic acid ion (RCOO—; R is a C₁₋₆ hydrocarbon group or H), and an alkoxide (RO⁻; R is a C₁₋₆ hydrocarbon group). Examples of the salt S of the present invention include optional combinations of the following cations and anions: the cation is one or more selected from an ammonium ion (NH₄⁺) and a sodium ion (Na⁺), and the anion is one or more selected from a chloride ion (Cl⁻), a formate ion (HCOO⁻), and an acetate ion (CH₃COO⁻). More specific examples of the salt S include ammonium chloride (NH₄Cl), ammonium formate (HCOONH₄), and sodium acetate (CH₃COONa).

### Type of Precursor of Salt

A precursor of a salt refers to an anion and a cation for forming a salt, and refers to a compound that is in a stage before the production of the anion or the cation. A precursor compound to produce a cation suitable for the object of the present invention is, for example, one or more selected from alkylammonium (NR¹R²R³; R¹ to R³ are each independently an alkyl group or H, and the alkyl group is preferably a C₁₋₆ hydrocarbon group), alkylsilazane (NR¹R²R³; R¹ to R³ are each independently an alkylsilyl group, an alkyl group, or H, and at least one is a silyl group), an ammine complex of a metal, a cyanamide, an alkali metal (particularly, Li, Na, K, Rb, Cs), and an alkaline earth metal (particularly, Mg, Ca, Sr, Ba). A precursor compound to produce an anion suitable for the object of the present invention is, for example, one or more selected from a hydrogen halide (particularly, HCl, HBr, HI), nitric acid (HNO₃), a sulfate ion (H₂SO₃), phosphoric acid (H₃PO₄), carbonic acid (H₂CO₃), an organic acid (RCOOH; R is an alkyl group or H, and the alkyl group is preferably a C₁₋₆ hydrocarbon group), and a metal alkoxide ((RO)ₙM; R is an alkyl group or H, and the alkyl group is preferably a C₁₋₆ hydrocarbon group; M is an alkali metal or an alkaline earth metal, preferably one or more selected from Li, Na, K, Rb, Cs, Mg, Ca, Sr, and Ba, and n is 1 or 2).

### Use of Titanium-Containing Silicon Oxide

The titanium-containing silicon oxide of the present invention can be used as a catalyst for oxidation reaction of an organic compound, for example, an epoxidation reaction of an olefin and is particularly preferably used for an epoxide production in which an olefin reacts with a hydroperoxide.

The olefin to be subjected to the epoxidation reaction may be an acyclic olefin, a monocyclic olefin, a bicyclic olefin or a polycyclic olefin having three or more rings, and may be a monoolefin, a diolefin or a polyolefin. When there are two or more double bonds in a molecule of the olefin, these double bonds may be a conjugated bond or a non-conjugated bond. The olefin having 2 to 60 carbon atoms is preferable. The olefin may have a substituent. Examples of such an olefin include ethylene, propylene, 1-butene, isobutylene, 1-hexene, 2-hexene, 3-hexene, 1-octene, 1-decene, styrene and cyclohexene. A substituent may be present in the olefin, the substituent containing an oxygen atom, a sulfur atom or a nitrogen atom together with a hydrogen atom or a carbon atom, or both of them. Examples of such an olefin include allyl alcohol, crotyl alcohol, and allyl chloride. Examples of the diolefin include butadiene and isoprene. Examples of the preferred olefin include an α-olefin. Examples of the particularly preferred olefin include propylene.

Examples of methods for producing propylene that is subjected to the epoxidation reaction include, but are not particularly limited to, cracking of naphtha or ethane; fluid catalytic cracking of vacuum gas oil; dehydrogenation of propane; disproportionation of ethylene and 2-butene: MTO (Methanol to Olefin) reaction to convert methanol or dimethyl ether; Fischer-Tropsch (FT) synthesis process to react carbon monoxide with hydrogen; and dehydration of isopropanol. Propylene, which is produced by a method that reduces the environmental burden, such as a method for obtaining propylene from bioethanol and/or isopropanol produced using a plant as a raw material; FT synthesis process using carbon dioxide or biomass; or a method of catalytic cracking of waste plastics, can also be used as a substrate for the epoxidation reaction.

Examples of the hydroperoxide include an organic hydroperoxide. The organic hydroperoxide is a compound having formula III,

R—O—O—H III
wherein R is a hydrocarbon group. The organic hydroperoxide reacts with the olefin to generate an epoxide and a hydroxyl compound. In formula III, R is preferably a hydrocarbon group having 3 to 20 carbon atoms and more preferably a hydrocarbon group having 3 to 10 carbon atoms. R may be linear or branched, and may be aliphatic or aromatic. Examples of the organic hydroperoxide include tert-butyl hydroperoxide, 1-phenylethyl hydroperoxide, and cumene hydroperoxide. Hereinafter, cumene hydroperoxide is sometimes abbreviated as CMHP.

When CMHP is used as the organic hydroperoxide, the obtained hydroxyl compound is 2-phenyl-2-propanol. Undergoing a dehydration reaction and a hydrogenation reaction of this 2-phenyl-2-propanol allows cumene to be generated. Hereinafter, cumene is sometimes abbreviated as CUM. Further, this CUM is oxidized, thereby obtaining CMHP again. From the viewpoint of these, CMHP is preferably used as an organic hydroperoxide used in the epoxidation reaction.

The epoxidation reaction can be carried out using a solvent, a diluent or a mixture thereof in the liquid phase. The solvent and the diluent need to be liquid under the temperature and pressure upon reacting and be substantially inactive to reactants and products. When CMHP is subjected to the epoxidation reaction in the presence of CUM which is a raw material thereof, CUM can also be used as a solvent without adding a solvent, especially.

The temperature of the epoxidation reaction is generally from 0 to 200° C. and is preferably a temperature from 25 to 200° C. The pressure of the epoxidation reaction can be a pressure sufficient to keep the reaction phase in a liquid state, and is generally preferably from 100 to 10000 kPa.

After finishing the epoxidation reaction, the liquid mixture containing a desired product can be separated from the titanium-containing silicon oxide. The liquid mixture can then be purified by a suitable method. Examples of the purification method include distillation, extraction and washing. The solvent and the unreacted olefin can be recirculated and reused.

The reaction using the titanium-containing silicon oxide produced according to one aspect of the present invention as a catalyst can be carried out in a form of a slurry or a fixed bed, and when a large-scale industrial operation is performed, the fixed bed is preferably used. When the titanium-containing silicon oxide produced according to one aspect of the present invention is used as a catalyst, the titanium-containing silicon oxide can be powder or a molded product. When being reacted in the fixed bed, the titanium-containing silicon oxide is preferably a molded product. This reaction can be carried out by a batch method, a semi-continuous method or a continuous method.

### EXAMPLES

Hereinafter, one aspect of the present invention will be described in further detail with reference to Examples.

### Example 1

### (1) Raw Material Mixing Step and Titanium Introducing Step

Hexadecyltrimethylammonium hydroxide (CTAH) which is diluted to a concentration of 16% by mass with a mixed solvent having a mixing ratio (mass ratio) of water:methanol=72:28 (125 parts by mass in a solution amount having a concentration of 16 mass % by mass) was stirred, then a mixed solution of 1.9 parts by mass of tetraisopropyl titanate and 4.35 parts by mass of 2-propanol was dropped thereto at room temperature under stirring. After the dropping was finished followed by stirring for 30 minutes, 30 g of tetramethyl orthosilicate was dropped under stirring. The stirring was then continued at room temperature for 3 hours, and the resulting solid was filtered. The obtained solid was dried under reduced pressure at 70° C. to obtain 34.8 parts by mass of a white solid. Hexadecyltrimethylammonium hydroxide, tetramethyl orthosilicate, and tetraisopropyl titanate are the template agent, the silicon source, and the titanium source, respectively.

To 15 parts by mass of the obtained white solid, water was added so that the water content might become 1.3 parts by mass, and they were well mixed. Thereafter, the obtained mixture was compression-molded. The obtained molded body was crushed, and the obtained crushed pieces were sieved to obtain 10 parts by weight of a molded body classified product containing the template agent and having a particle diameter of 1.0 to 2.0 mm.

### (2) Template Agent Removing Step

A cylindrical glass column having an inner diameter of 30 mm (sleeve tube outer diameter 8 mm) and a height of 27 cm, which had been vertically installed, was packed with 20 g of the molded body obtained above. At that time, a packing length of the molded body was 6.3 cm. Thereafter, the following 3 solutions were successively passed through the column upward from the bottom of the column. First, 141 g of methanol was passed through at a column temperature of 25° C. and a liquid passing rate=3.5 g/min. Next, a mixed solution of 326 g of methanol and 8 g of concentrated hydrochloric acid (hydrogen chloride content 36 mass %) was passed through at a column temperature of 38° C. and a liquid passing rate=3.0 g/min. Next, 190 g of methanol was passed through at a column temperature of 38° C. and a liquid passing rate=3.5 g/min, and thereafter, with cooling the column to 25° C., 126 g of methanol was passed through at a liquid passing rate=3.5 g/min. After this, a solution containing the template agent and methanol in the column was drawn out from the bottom or the column.

Subsequently, 47 g of toluene was passed through the column at a column temperature of 75° C. and a liquid passing rate=2.8 g/min, and thereafter, with raising the column temperature up to 90° C., 157 g of toluene was passed through the column. Due to this, the mixed solution remaining in the column at the end of the template agent removing step was replaced with toluene. Thereafter, toluene in the column was drawn out from the bottom of the column. Thereafter, nitrogen gas was allowed to flow in the column upward from the bottom of the column at a column temperature of 120° C. and a flow rate of 50 NmL/min, then that the distillation of the liquid from the top of the column had stopped was confirmed, thereafter the flow rate was changed to 150 NmL/min, and nitrogen gas was passed through the column for 2 hours in total to dry the molded body. Thus, 8 g of a solid was obtained.

### (3) Silylation Step

8 g of the solid obtained in the template agent removing step, 8 g of trimethylsilylchloride, and 80 g of toluene were mixed, then the mixture was heated to reflux for 1.5 hours. The mixture was allowed to cool and the solid was then filtered. The obtained solid was washed with 30 g of toluene and dried under reduced pressure at 120° C. and 10 mmHg for 2 hours, thereby obtaining a titanium-containing silicon oxide catalyst.

### (4) Salt Concentration Adjusting Step

To 10 g of the solid obtained in the silylation step, 100 g of a methanol solution in which 0.03 g of ammonium chloride had been dissolved was added, and the mixture was stirred at room temperature for 1 hour. Then, after the solvent was removed under reduced pressure at 40° C. and 50 mmHg using a rotary evaporator, drying was performed at 60° C. and 10 mmHg for 2 hours to obtain 10 g of a titanium-containing silicon oxide.

The catalytic performance evaluation was carried out by the method described below.

### (5) Evaluation of Catalytic Performance

Performance of the titanium-containing silicon oxide obtained through the above steps (1) to (4) was evaluated by a batch type reactor (autoclave). To the autoclave, 0.5 g of the titanium-containing silicon oxide, 60 g of a solution in which CMHP had been dissolved in CUM at a concentration of 25 mass % (referred to as 25 mass % CMHP/CUM hereinafter), and 33 g of propylene were fed, and they were reacted under autogenous pressure at a reaction temperature of 100° C. for a reaction time of 1.5 hours (including heating time). The reaction results are set forth in Table 1.

The “CMHP conversion rate”, “PO selectivity rate”, and “PGs (polypropylene glycols) selection rate” were determined as follows.

$$CMHP conversion rate ( \% ) = {M}_{2} / {M}_{0} \times 100$$

M₀: Molar amount of raw material CMHP
M₁: Molar amount of CMHP in liquid after reaction
M₂: Molar amount of CMHP reacted
in this regard, M₂=M₀−M₁;

$$PO selection rate ( \% ) = {M}_{PO} / {M}_{2} \times 100$$

M_{PO}: Molar amount of PO generated
in this regard, M_{PO}=M₂−(Mₚₕ+M_{ac}+M_{pg}+2×M_{dpg}+3×M_{tpg}),
Mₚₕ: Molar amount of phenol generated
M_{ac}: Molar amount of acetophenone generated
M_{pg}: Molar amount of propylene glycol generated
M_{dpg}: Molar amount of dipropylene glycol generated
M_{tpg}: Molar amount of tripropylene glycol generated;

$$PGs selection rate ( \% ) = {M}_{PGs} / {M}_{2} \times 100$$

M_{PGs}: Molar amount of PGs generated
in this regard, M_{PGs}=M_{pg}+2×M_{dpg}+3×M_{tpg}.

### (6) Evaluation of Pore Structure

In order to evaluate the pore structure of the titanium-containing silicon oxide obtained through the above steps (1) to (4), pretreatment by vacuum heating deaeration at 120° C. for 2 hours using BELSORP MINI X, manufactured by MicrotracBEL Corp., followed by carrying out nitrogen adsorption measurement of the titanium-containing silicon oxide, to determine an average pore size, a total pore volume, and a total volume of pores of 5 to 200 Å by pore distribution calculation due to BJH method. Further, a ratio of the total volume of pores of 5 to 200 Å to the total pore volume was determined. The pore structure is set forth in Table 2.

### Examples 2 to 5 and Comparative Examples 1 and 2

Regarding Examples 2 to 5 and Comparative Examples 1 and 2, the above steps (1), (2), and (3), and the evaluations (5) and (6) were carried out in the same manner as in Example 1. The step (4) was carried out in the same manner as in Example 1 except that the amount of ammonium chloride added was changed as described in Table 1.

TABLE 1

| | Amount of | Ammonium | CMHP | PO | PGs |
|---|---|---|---|---|---|
| | ammonium | chloride/Ti | con- | selec- | selec- |
| | chloride | molar | version | tion | tion |
| | added | ratio | rate | rate | rate |
| | (g) | (—) | (%) | (%) | (%) |
| Example 1 | 0.027 | 0.199 | 83.10 | 98.62 | 1.01 |
| Example 2 | 0.001 | 0.004 | 83.95 | 98.17 | 1.21 |
| Example 3 | 0.005 | 0.040 | 82.90 | 98.27 | 1.14 |
| Example 4 | 0.134 | 0.997 | 82.40 | 98.81 | 0.89 |
| Example 5 | 0.535 | 3.989 | 83.58 | 98.78 | 0.93 |
| Comparative | 0.000 | — | 83.40 | 98.00 | 1.43 |
| Example 1 | | | | | |
| Comparative | 2.675 | 19.945 | 46.13 | 98.76 | 0.85 |
| Example 2 | | | | | |

TABLE 2

| | | | Total | Ratio of total |
|---|---|---|---|---|
| | Average | Total | volume of | volume of pores |
| | pore | pore | pores of 5 | of 5 to 200 Å to |
| | size | volume | to 200 Å | total pore volume |
| | (Å) | (ml/g) | (ml/g) | (%) |
| Example 1 | 24.2 | 1.05 | 1.02 | 97.1 |
| Example 2 | 24.5 | 1.09 | 1.04 | 95.4 |
| Example 3 | 24.4 | 1.12 | 1.05 | 93.8 |
| Example 4 | 24.2 | 1.11 | 1.05 | 94.6 |
| Example 5 | 23.4 | 1.01 | 0.95 | 94.1 |
| Comparative | 24.5 | 1.11 | 1.04 | 93.7 |
| Example 1 | | | | |
| Comparative | 20.3 | 0.85 | 0.81 | 95.3 |
| Example 2 | | | | |

### Examples 6 and 7

Regarding Examples 6 and 7, the above steps (1), (2), and (3), and the evaluations (5) and (6) were carried out in the same manner as in Example 1. The step (4) was carried out in the same manner as in Example 1 except that the type (ammonium chloride) and the concentration of the salt described in Table 1 were changed as described in Table 3.

TABLE 3

| | | Amount | Salt/Ti | CMHP | PO | PGs |
|---|---|---|---|---|---|---|
| | | of salt | molar | conversion | selection | selection |
| | | added | ratio | rate | rate | rate |
| | Type of salt | (g) | (—) | (%) | (%) | (%) |
| Example 6 | Ammonium formate | 0.032 | 0.199 | 81.84 | 98.47 | 1.18 |
| Example 7 | Sodium acetate | 0.041 | 0.199 | 83.41 | 99.05 | 0.50 |

TABLE 4

| | | | Total | Ratio of total |
|---|---|---|---|---|
| | Average | Total | volume of | volume of pores |
| | pore | pore | pores of 5 | of 5 to 200 Å to |
| | size | volume | to 200 Å | total pore volume |
| | (Å) | (ml/g) | (ml/g) | (%) |
| Example 6 | 24.6 | 1.07 | 0.99 | 92.5 |
| Example 7 | 24.1 | 1.08 | 1.01 | 93.5 |

### INDUSTRIAL APPLICABILITY

The method for producing a titanium-containing silicon oxide according to one aspect of the present invention can be applied to a production of a catalyst which can be used in a reaction of generating an epoxide from an olefin and a hydroperoxide, and the titanium-containing silicon oxide obtained by the method can be used, for example, as a catalyst, in a production of a propylene oxide.

1.-7. (canceled)

8. A titanium-containing silicon oxide satisfying all conditions 1 to 5:
condition 1: an average pore size is 10 Å or more;
condition 2: pores of 80% or more of a total pore volume each have a pore size of 5 to 200 Å;
condition 3: the total pore volume is 0.2 cm³/g or more;
condition 4: the titanium-containing silicon oxide is obtained by using a quaternary ammonium ion represented by formula I as a template agent and thereafter removing the template agent by a solvent extraction operation:
[NR′R²R³R⁴]⁺ I
wherein R¹ represents a C₂₋₃₆ hydrocarbon group, and R² to R⁴ each independently represent a C₁₋₆ hydrocarbon group;
condition 5: a ratio of an amount of substance of a salt S to an amount of substance of titanium atoms in the titanium-containing silicon oxide is 0.004 to 10, and the salt S is ammonium chloride.

9. A method for producing an epoxide from an olefin, the method comprising using the titanium-containing silicon oxide according to claim 8.

10. A method for producing a titanium-containing silicon oxide, comprising the following steps:
a step of mixing a silicon source, a template agent, and a solvent to obtain a solid including a silicon oxide and the template agent (raw material mixing step);
a step of removing the template agent from the solid obtained in the raw material mixing step to obtain a solid including the silicon oxide (template agent removing step);
a step of contacting the solid obtained in the template agent removing step with a silylating agent to obtain a solid including a silylated silicon oxide (silylation step);
a step of introducing titanium into the system (titanium introducing step); and
a step of introducing or removing a salt S or a precursor thereof into the system to adjust a molar concentration of the salt S or a precursor thereof relative to an amount of substance of titanium atoms into the system, wherein the salt S is ammonium chloride (salt concentration adjusting step).

11. A method for producing an epoxide, comprising a step of reacting an olefin with a hydroperoxide in the presence of the titanium-containing silicon oxide according to claim 8.

12. The method according to claim 11, wherein the olefin is propylene.

13. The method according to claim 11, wherein the hydroperoxide is cumene hydroperoxide.

14. The method according to claim 12, wherein the hydroperoxide is cumene hydroperoxide.
